(19) United States
(12) Patent Application Publication
(10) Pub. No.: US 2019/0021750 A1
(43) Pub. Date: Jan. 24, 2019

(54) Methods and Systems for Removing Clots from Blood Vessels

(71) Applicant: Cerevasc, LLC, Boston, MA (US)

(72) Inventors: Carl Heilman, Wayland, MA (US); Adel M. Malek, Weston, MA (US); David A. Rezac, Westborough, MA (US)

(73) Assignee: Cerevasc, LLC, Boston, MA (US)

(21) Appl. No.: 16/067,486

(22) PCT Filed: Dec. 29, 2016
(86) PCT No.: PCT/US16/69280

§ 371 Date: Jun. 29, 2018

Related U.S. Application Data
(60) Provisional application No. 62/273,934, filed on Dec. 31, 2015.

(51) Int. Cl. A61B 17/221

(52) U.S. Cl. CPC A61B 17/221; A61B 2017/00862

(57) ABSTRACT

This disclosure relates to systems, devices, and methods for removing clots, e.g., calcified clots, from a blood vessel and/or for removing a calcified clot from a blood vessel wall. The devices include an elongated tubular body including a wall surrounding a central lumen and having proximal and distal ends. The tubular body includes at least one set of two, three, or more elongated cuts through the wall to form a clot engaging member having two, three, or more ribs between the cuts, wherein a first set of elongated cuts is arranged adjacent to the distal end of the body, and wherein the clot engaging member is made of an elastic material configured to cause the ribs to self-expand radially outwardly from the tubular body when the body is in a non-constrained, deployed configuration.

### FIELD OF THE INVENTION

The present disclosure pertains generally to methods and systems for clots from blood vessels, e.g., for stroke treatment. More particularly, the present disclosure pertains to methods and systems for treating acute ischemic stroke by mechanical thrombectomy.

### BACKGROUND

The World Health Organization has reported that 15 million people worldwide suffer a stroke each year; and further, that of these 15 million stroke victims, approximately five million die from the event and another five million are left permanently disabled. There are two types of stroke: ischemic and hemorrhagic. With an ischemic stroke, a blockage in the arterial vasculature, typically caused by a clot or plaque build up in the vessel, prevents blood flow to certain portions of the brain. Hemorrhagic stroke is characterized by a ruptured wall in the arterial vasculature, which causes bleeding into the surrounding tissues and prevents blood flow to certain portions of the brain. Nearly 90 percent of all strokes are ischemic events, with the remainder classified as hemorrhagic.

Treatment options for acute ischemic stroke include intravenous administration of fibrinolytic or “clot-busting” compounds (e.g., recombinant tissue plasminogen activator or “rtPA”), and endovascular intervention using mechanical thrombectomy devices. Mechanical thrombectomy devices encompass a wide array of endovascular tools designed to remove clots or thrombi from the vasculature in acute ischemic stroke patients. Such devices include coil retrievers (e.g., deploying a coil made from a shape-memory material across a clot to engage the thrombus, and then pulling the coil-engaged clot into the delivery catheter), aspiration devices (e.g., applying vacuum aspiration in a vessel to remove an occlusive clot), stent retrievers or “stentrievers” (e.g., deploying a self-expanding stent in the occluded portion of the vessel to engage the clot and then withdrawing the stent and clot from the vessel into the delivery catheter), and snares.

In 2015, the American Heart Association and American Stroke Association (AHA/ASA) issued a focused update to prior guidance for the endovascular treatment of acute ischemic stroke. The AHA/ASA focused update recommends that acute ischemic stroke patients eligible for rtPA treatment should receive such therapy, and endovascular treatment with a stent retriever provided the patient, among other criteria, has an occlusion in the internal carotid artery (ICA) or middle cerebral artery (MCA). The AHA/ASA update is based, in part, on recent clinical studies demonstrating significantly improved outcomes for ischemic stroke patients treated with rtPA and stent retrievers compared to rtPA alone. While the AHA/ASA update specifically focuses on stent retrievers used in the ICA or MCA, stent retrievers are commonly used to treat acute ischemic stroke in other vessels such as the anterior cerebral artery, vertebral artery, and basilar artery.

Several stent retriever devices are available to clinicians for treating acute ischemic stroke. For example, Stryker Neurovascular offers a Trevo® XP Provue Retriever, and Medtronic's Minimally Invasive Therapies Group offers the Solitaire® FR Revascularization Device. These and other devices intended to treat acute ischemic stroke, however, may not be suitable for all ischemic stroke events, particularly those caused by severely calcified or partially calcified clots (e.g., non-fresh or non-soft thrombus as compared to soft or fresh thrombus typically associated with, for example, ischemic stroke resulting from atrial fibrillation).

### SUMMARY

Embodiments of the present disclosure include devices and methods for removing calcified clots from blood vessels. In one aspect the disclosure features systems for removing a calcified clot from a blood vessel. The systems include a clot retrieval device including an elongated tubular body comprising a wall surrounding a central lumen and having proximal and distal ends, wherein the elongated tubular body includes at least one set of two or more, e.g., three, four, five, or more, elongated longitudinal cuts through the wall to form a clot engaging member including two or more, e.g., three, four, five, or more, deformable ribs between the cuts, wherein a first set of elongated longitudinal cuts is arranged adjacent to the distal end of the elongated tubular body, and wherein the ribs comprise or consist of an elastic material, e.g., a super-elastic and/or shape-memory material, configured to cause the ribs to self-expand radially outwardly from the body when the clot engaging member is in a non-constrained, deployed configuration and wherein the deployed, expanded ribs provide sufficient resistive force such that the ribs do not break or resume a constrained delivery configuration when engaging a hardened, calcified clot; and a pusher element coupled to a proximal portion of the clot retrieval device and configured to enable a user to manipulate, e.g., move, the device distally and proximally, e.g., push or pull the device, within a blood vessel. The systems can further include a delivery catheter configured to contain the clot retrieval device in a constrained delivery configuration.

As used herein, the distal end of the device is inserted into a patient first and the proximal end is closest to the operator, e.g., surgeon or electro-mechanical system, of the device.

In certain implementations, the tubular body can include cuts or slots or recesses arranged proximally of the clot engaging member to provide greater flexibility to the tubular body.

In various embodiments, the tubular body can include a second set of elongated cuts arranged proximally from the first set of elongated cuts to form a second clot engaging member, wherein a body portion separates the first and second sets of cuts.

In any of the implementations described herein, the elastic material can be or comprise a metal or a metal alloy. For example, the metal alloy can be or include a shape-memory alloy and/or super-elastic alloy that returns to a predetermined shape when in a non-constrained, expanded, deployed configuration. In some implementations the metal alloy comprises nickel and titanium, e.g., as in nitinol.

The elongated cuts can be arranged in parallel, can be radially spaced apart at equal distances between them, or can be both arranged in parallel and radially spaced apart at equal distances between them.

In some embodiments the pusher element can be a detachable wire or a non-detachable wire. In some implementations the pusher element and the tubular body are formed from a single piece of material.

In certain embodiments the tubular body between the first and second clot engaging members includes or is formed as a biased or preformed coil shape that tensions the first and second clot engaging members towards each other, e.g., once the clot retrieval device is in a non-constrained, deployed configuration or depending on operator manipulation of the device. In some implementations the tubular body portion includes cuts or slots arranged to provide greater flexibility to the body portion. In various implementations the exterior portions of the first and/or second clot engaging member(s) have a curved, atraumatic profile.

In another aspect, the disclosure features methods of removing a calcified clot from a blood vessel in a subject. These methods include guiding a delivery catheter through a blood vessel in the subject to a location in the blood vessel distally beyond the calcified clot, wherein the delivery catheter contains a clot removal system as described herein and wherein the at least one clot engaging member is in a constrained delivery configuration within the delivery catheter; using the pusher element to advance the clot retrieval device partially out of the delivery catheter while maintaining the at least one clot engaging member in a constrained delivery configuration; withdrawing the delivery catheter while maintaining the clot retrieval device in position within the blood vessel with the pusher element to advance the clot retrieval device out of the delivery catheter such that at least one clot engaging member self-expands into a non-constrained, deployed configuration; manipulating the clot retrieval device to cause ribs of the at least one clot engaging member in the non-constrained, deployed configuration to contact the clot from a location distally beyond the clot; and withdrawing the clot retrieval device from the location to pull the clot along with the at least one clot engaging member in the non-constrained, deployed configuration until the clot has been removed from the blood vessel.

These methods can further include guiding a guide wire through the blood vessel in the subject to a location in the blood vessel distally beyond the calcified clot; and guiding the delivery catheter along the guide wire to a location in the blood vessel distally beyond the calcified clot.

In any of these methods, withdrawing the clot retrieval device can include pulling the clot retrieval device into the delivery catheter such that the clot is also pulled into the delivery catheter. Alternatively, withdrawing the clot retrieval device can include pulling the clot retrieval device to a distal end of the delivery catheter such that the clot is secured between the distal end of the delivery catheter and one or more ribs of the at least one clot engaging member in the deployed configuration.

In some implementations of these methods the clot retrieval device includes at least two clot engaging members and wherein the clot retrieval device is manipulated such that the first clot engaging member is deployed distally beyond the clot and the second clot engaging member is deployed proximally of the clot such that the clot is located between the first and second clot engaging members.

In various implementations the body portion of the clot retrieval device can include a biased or preformed coil shape that tensions the first and second clot engaging members towards each other and then further includes manipulating the biased or preformed coil shape to cause the first and second clot engaging members to secure the clot between them.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes, e.g., (1.0, 1.5, 2.0, 2.75, 3.0, 3.80, 4.0, 4.5, and 5.0).

As used in this specification and the appended claims, the singular forms “a,” “an,” and “the” include plural referents unless the content clearly dictates otherwise. As used in this specification and the appended claims, the term “or” is generally employed in its sense including “and/or” unless the content clearly dictates otherwise.

Various embodiments are described hereinafter with reference to the figures. The figures are not necessarily drawn to scale (unless indicated), the relative scale of select elements may have been exaggerated for clarity, and elements of similar structures or functions are represented by like reference numerals throughout the figures. It should also be understood that the figures are intended merely to facilitate the description of the embodiments, and are not intended as an exhaustive description of the invention or as a limitation on the scope of the invention, which is defined only by the appended claims and their equivalents. In addition, an illustrated embodiment needs not include all the aspects or advantages shown. An aspect or an advantage described in conjunction with a particular embodiment is not necessarily limited to that embodiment and can be practiced in any other embodiments even if not so illustrated.

References herein to the term “endovascular,” such as endovascular technique or endovascular approach, generally refer to minimally-invasive devices, systems, and procedures configured for introduction into a patient's vasculature (e.g., femoral artery) through a small access device (e.g., needle or introducer sheath) without a large incision or open surgical procedure, and using the vasculature to guide various catheters, guide wires, clot retrievers, and other system elements described herein percutaneously to a target procedural location disposed within the patient's vasculature (e.g., anterior cerebral artery, posterior cerebral artery, middle cerebral artery, vertebral artery, basilar artery).

The term “self-expand” refers to clot engaging elements, such as ribs, that are made of an elastic material, e.g., a super-elastic and/or shape-memory metal alloy, that is configured to retain an expanded configuration once any constraint is removed, e.g., when the device including the clot engaging member(s) is pushed out of a delivery catheter that is configured to compress and constrain the ribs of the clot engaging member of the clot retrieval device in a constrained, narrow cross-sectional diameter, delivery configuration. Once released from the constraint, the ribs automatically spread radially outwardly to an increased cross-sectional diameter when deployed in a blood vessel without any further manipulation or input of energy required by the user. The constraint can be a physical, e.g., mechanical, constraint, but in some embodiments can be a temperature or other constraint. For example, a clot engaging member may have a constrained delivery configuration at room temperature, and an expanded, non-constrained deployed configuration at body temperature.

Unless otherwise defined, all technical and scientific terms used herein have the same meaning as commonly understood by one of ordinary skill in the art to which the subject matter of this disclosure belongs. Although methods and materials similar or equivalent to those described herein can be used in the practice or testing of the implementations described herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages will be apparent from the following detailed description, and from the claims.

### BRIEF DESCRIPTION OF THE DRAWINGS

FIG. 1 is a schematic diagram of a patient's neurovascular anatomy.

FIG. 2 is a side view of a clot retrieval device according to one of the embodiments disclosed herein.

FIGS. 3A-3S are additional exemplary cut patterns for forming a clot retrieval device, according to additional embodiments disclosed herein.

FIG. 4 is a side view of a clot retrieval device according to another embodiment disclosed herein.

FIGS. 5A-5B are a side view and perspective view, respectively, of a clot retrieval device according to another embodiment disclosed herein.

FIGS. 6A-6C are schematics of examples of cut patterns for forming a clot retrieval device according to additional embodiments disclosed herein. FIG. 6D is a close-up view of an interlocking element.

FIGS. 7A and 7B are side and perspective views, respectively, of a clot retrieval device according to one of the embodiments disclosed herein.

FIGS. 8A and 8B are side views of a middle portion of a clot retrieval device having a spring-like configuration in an expanded (8A) and a compressed (8B) state according to another embodiment disclosed herein.

FIG. 9 is a side view of a clot retrieval device according to another embodiment disclosed herein.

FIG. 10 is a perspective view of a clot retrieval device according to another embodiment disclosed herein.

FIG. 11 is a side view of a clot retrieval device according to another embodiment disclosed herein that includes a mesh structure.

FIG. 12 is a side view of a clot engaging member according to one of the embodiments disclosed herein.

FIG. 13 is a side view of a clot retrieval device according to another of the embodiments disclosed herein including radiopaque markings.

FIGS. 14A-14D are cross-sectional views of delivery catheters according to additional embodiments disclosed herein.

FIGS. 15A-15E are side views of a clot retrieval device as used, for example, in the methods described herein.

FIGS. 16A-16D are side views of a clot retrieval device according to another one of the embodiments disclosed herein as used, for example, in the methods disclosed herein.

### DETAILED DESCRIPTION

Clot retrieval device embodiments disclosed herein, also referred to herein as clot retrievers, advantageously engage and remove calcified clots, e.g., those causing an ischemic stroke event. The overall flexibility and navigability of the new devices assists with delivery of the clot retriever to the clot in the patient's vasculature through a delivery catheter. Features of the clot retriever embodiments, including clot engaging members, are capable of engaging a calcified clot for retrieval and removal from the patient (e.g., due, in part, to the high radial expansion force and sufficiently robust construction of clot engaging members). As further described below, clot retriever embodiments comprise super-elastic materials (e.g., nickel-titanium alloys such as nitinol) that add to the overall device flexibility, while preserving sufficient column strength to engage and remove a calcified clot from a vessel lumen. In addition, the configuration of the clot engaging member(s) including the dimensions of the deployed, self-expanded ribs provide sufficient resistive force such that the clot engaging members do not break or resume a constrained delivery configuration when engaging a hardened, calcified clot as is typically seen with other mechanical thrombectomy devices. Exterior portions of the clot engaging members have a curved, atraumatic profile to minimize damage to the arterial walls when deployed in a patient's vessel, thereby minimizing the risk of complications to the patient while the clinician engages and removes the stroke-causing calcified clot.

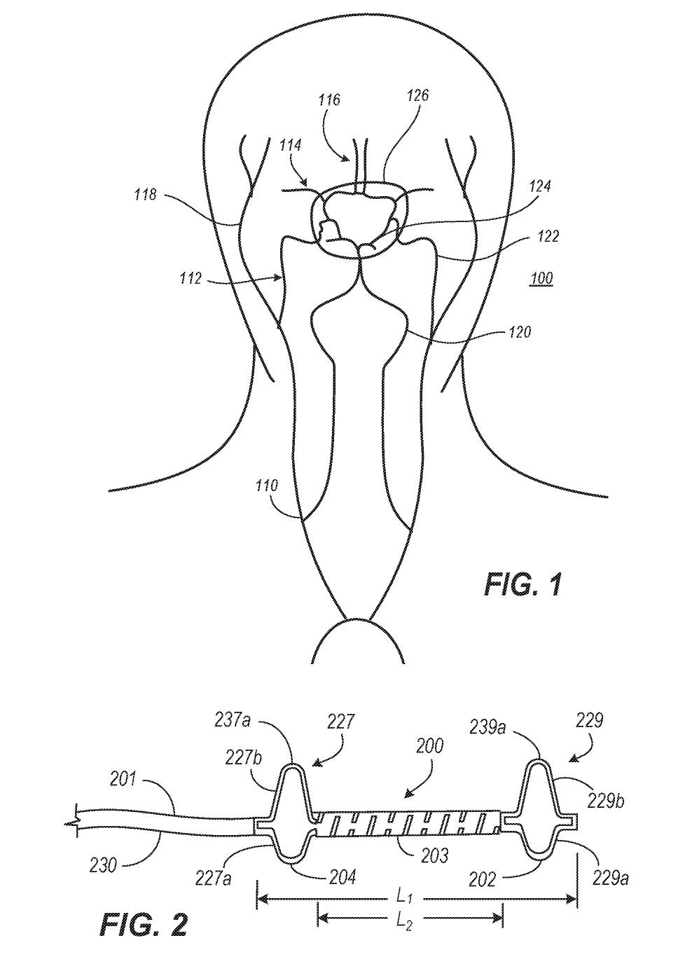

FIG. 1 is a schematic diagram of a patient's neurovascular anatomy 100 where embodiments of a clot retriever 200 disclosed herein can be deployed to treat acute ischemic stroke resulting from, for example, a calcified clot (as defined herein, a calcified clot can be partially or severely calcified, but is not a “fresh” or “soft” thrombus typically seen with acute ischemic stoke following atrial fibrillation); clot retriever 200 is used to remove the calcified clot obstructing blood flow in an arterial vessel. The arterial neurovascular anatomy shown in FIG. 1 includes carotid artery 110, internal carotid artery 112 (ICA), middle cerebral artery 114 (MCA), and anterior cerebral artery 116 (ACA). An external carotid artery 118 is also shown in FIG. 1. The internal diameter of the ICA can range from about 2.5 mm to about 5 mm. The MCA internal diameter can range from about 1.5 mm to about 4.5 mm. The carotid and cerebral arterial structures (e.g., 110, 112, 114, 116, and 118) comprise a portion of the anterior arterial blood circulation pathway in a patient's neurovascular anatomy 100. The posterior arterial blood circulation pathway includes the vertebral artery 120, basilar artery 122, and posterior cerebral artery 124 (PCA) shown in FIG. 1.

FIG. 2 depicts one embodiment of a clot retrieval device 200 intended to treat ischemic stroke patients by engaging and retrieving a calcified clot in a patient's arterial vasculature, thereby restoring arterial blood flow. Clot retriever 200 includes at least one clot engaging member 229 in the distal portion 202 of the device, and optionally a second clot engaging member 227 in the proximal portion 204 of the device and an elongate body, e.g., a tubular body portion 203, e.g., extending between the respective clot engaging members. The clot engaging members 227 and 229 of the FIG. 2 embodiment comprise a plurality of respective deformable elements (e.g., ribs) 227a, 227b, and 227c (227c not shown) and 229a, 229b, and 229c (229c not shown) that are disposed radially outwardly in the deployed, unconstrained configuration. In the deployed configuration, the diameter of the clot engaging members (e.g., diameter across the expanded clot engaging member) can range from about 1 mm to 5 mm or more.

In some embodiments, clot retriever 200 can be connected to a pusher element 230 (e.g., a detachable or non-detachable wire made of, e.g., stainless steel, titanium, nitinol or other nickel titanium alloys) around transition portion 201 of the device as shown in FIG. 2, or formed from a single piece of material that encompasses the device 200 and pusher element 230. In any of these configurations, the pusher element 230 extending from clot retriever 200 has sufficient length (e.g., 150 cm, 180 cm, or more) to extend proximally from device 200 deployed in the patient's arterial vasculature described with FIG. 1, e.g., to the femoral artery, and out through the access location, e.g., in the patient's groin. The length is sufficient, and the strength and dimensions of the material are sufficient, such that a clinician can use pusher element 230 to manipulate or navigate a device 200 through a delivery catheter when treating an ischemic stroke, as will be further described below. The “pusher element” 230 is configured so that it can be used to both advance (push) the clot retrieval device 200 into and within a subject's blood vessels without buckling, and to remove (pull) the device within and out of the blood vessels without breaking.

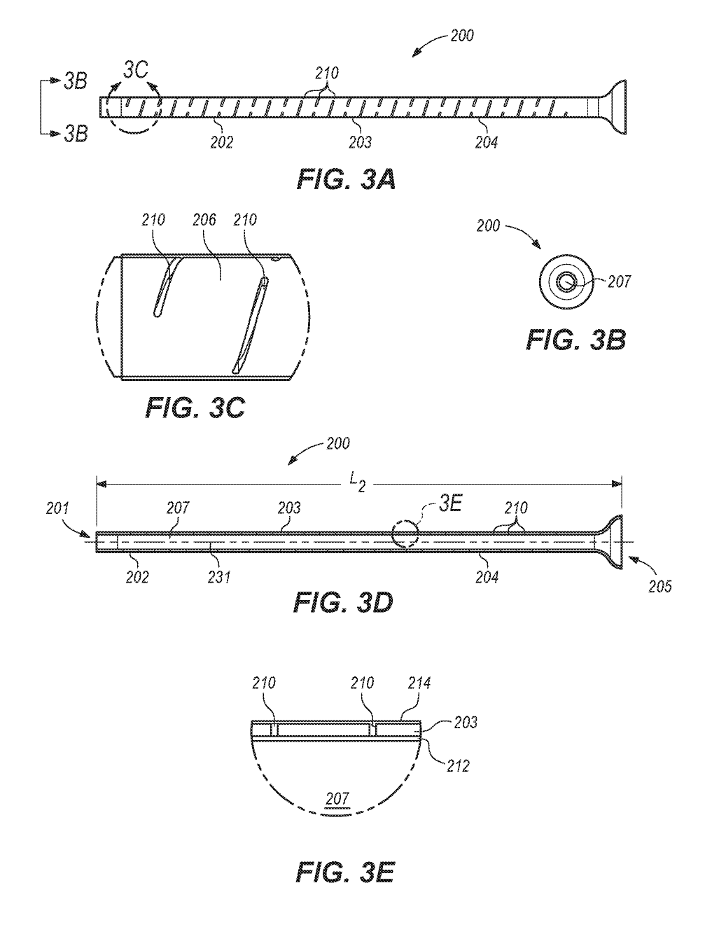

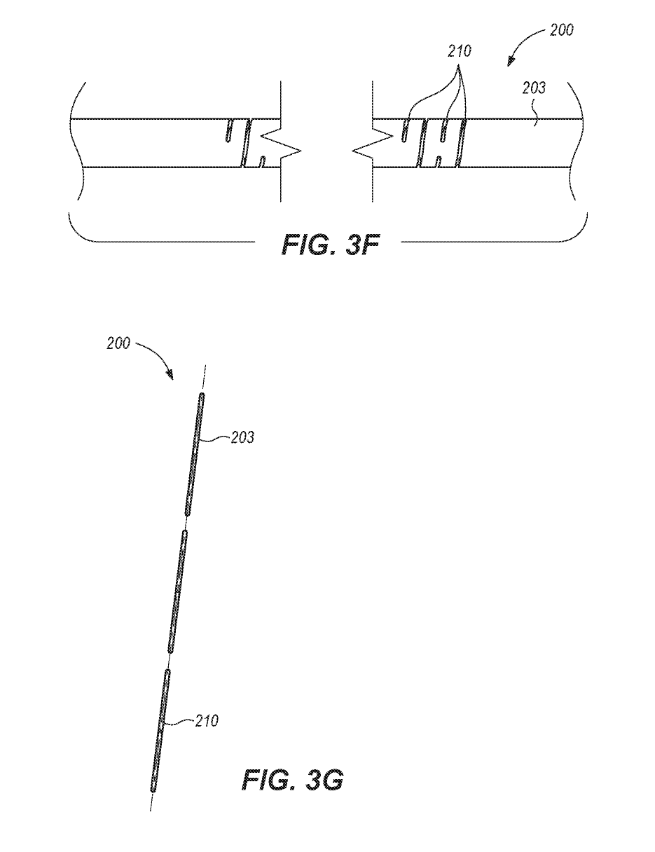

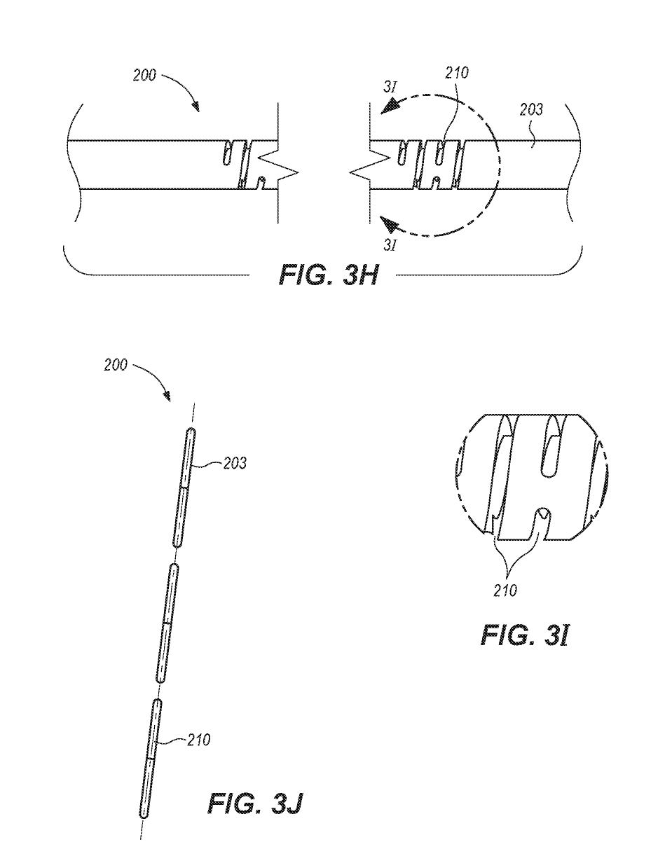

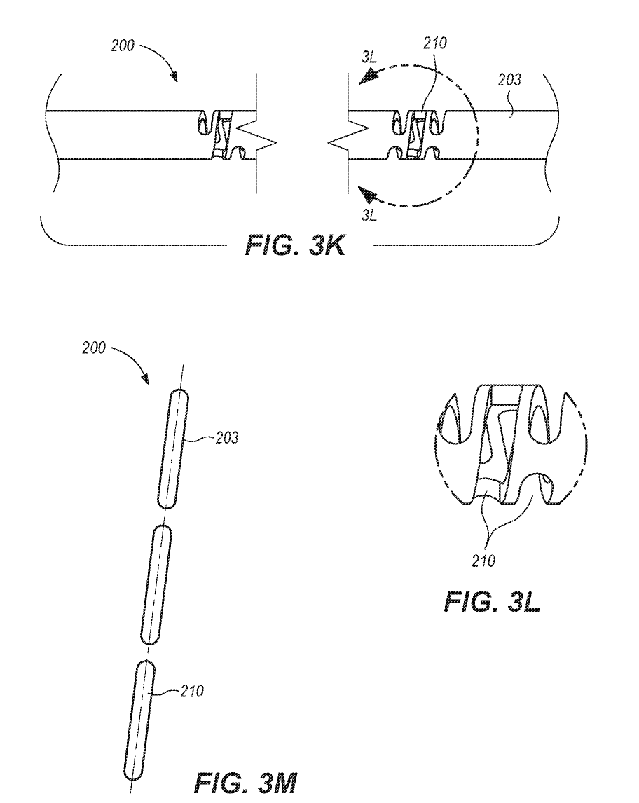

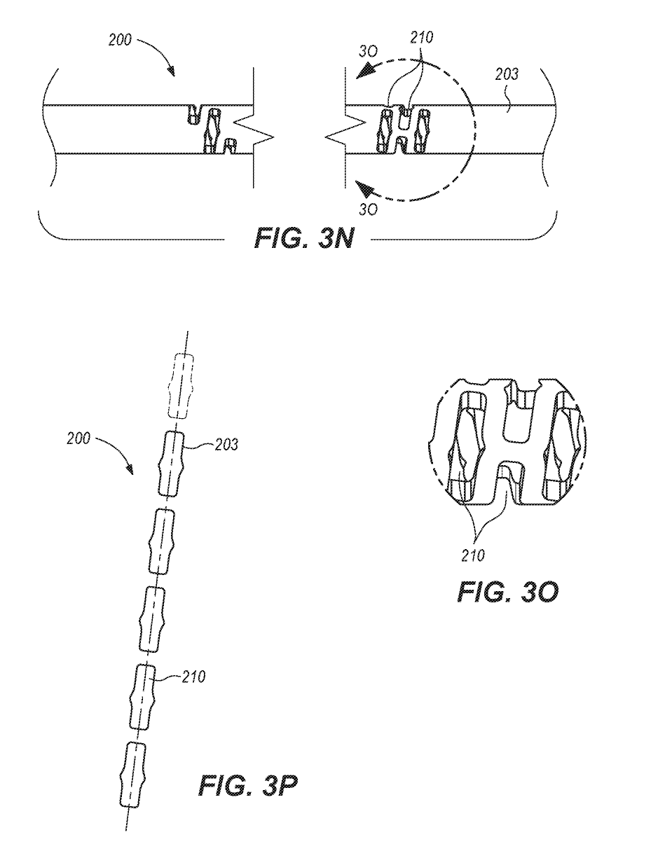

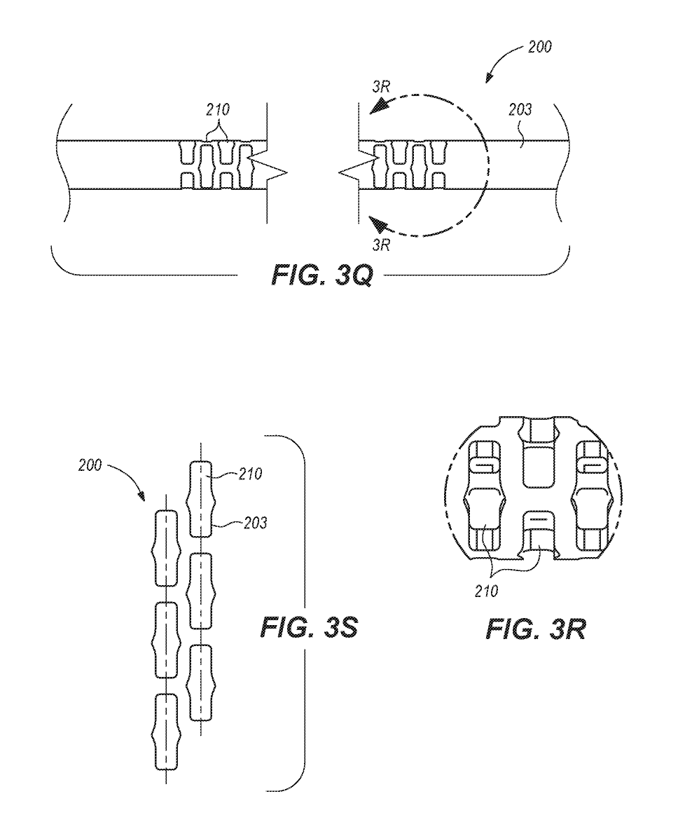

Clot retriever 200 and clot engaging members 227, 229 can be composed from any number of biocompatible, constrainable, elastic materials or combinations thereof, including metals, and metal alloys, such as stainless steel, tantalum, or super-elastic metal alloys, e.g., nickel titanium alloys known as nitinol. The clot retriever 200, particularly the body portion 203 of the various clot retriever 200 embodiments shown in FIGS. 3A-3S, can be composed of nitinol or other nickel titanium alloys comprising copper, cobalt, chromium, and/or vanadium. Clot engaging members 227 and 229 can have a preformed, expanded, deployed configuration (e.g., configuration shown in FIG. 2), for example, when constructed from super-elastic materials such as nitinol. In addition, construction from super-elastic materials allows for clot retriever 200 and clot engaging members 227, 229 to be compressed or constrained radially about the longitudinal axis of the clot retriever for navigation within a delivery catheter (e.g., a micro catheter, such as an 0.027,″ 0.021,″ 0.018″ or other size micro catheter) through the patient's vasculature. The preformed, super-elastic materials also provide a self-expanding feature of the clot engaging members 227 and 229 (e.g., causes the clot engaging members to assume a deployed configuration as they emerge from the confines of the lumen of a delivery catheter).

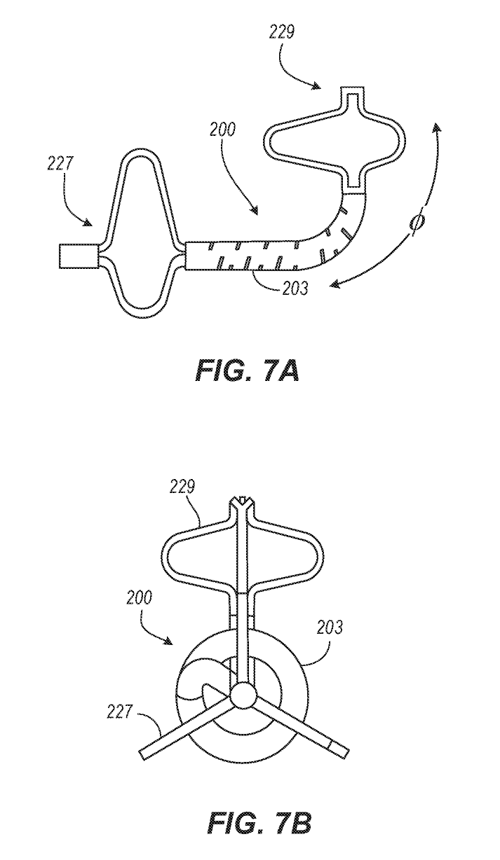

As shown in FIG. 3A, clot retriever 200 further comprises one or more cuts 210 (e.g., slots, kerfs, key-ways, recesses, or the like) along the body portion 203. The cuts 210 of the body portion 203 are configured to increase the flexibility of the clot retriever 200 for navigating tortuous anatomy during delivery and/or to assume a pre-determined configuration (e.g., secondary shape, for example, a bend or coil shape between proximal portion 204 and distal portion 202 as shown in FIGS. 7A, 7B when deployed to engage clot 130). The cuts 210 of the body portion 203 can have a variety of suitable patterns, e.g., as shown in FIGS. 3A-3S.

The cuts 210 and their patterns can be manufactured by cutting, e.g., by laser cutting, a hypo-tube of super-elastic alloy to form the body portion 203 of the clot retriever 200. Alternatively, the cuts 210 and their patterns can be manufactured by etching, stamping, water jet, or other suitable techniques. The hypo-tube can comprise nitinol, can have an inner diameter ranging from about 0.004 inches to about 0.020 inches or more, and can have a wall thickness from about 0.0012 inches to about 0.0150 inches or more. In the embodiment of clot retriever 200 shown in FIG. 3A-3E, each cut 210 in hypo-tube 206 can have a width of 0.001 inches (0.0254 mm). The width, length, and depth of each cut 210 and patterns in the body portion 203 of the clot retriever 200, can comprise any suitable dimensions.

As best shown in FIGS. 3B and 3D, the clot retriever 200 can include a lumen 207 extending from a proximal opening 205 in the proximal portion 204 to a distal opening in transition portion 201 in the distal portion 202 of the clot retriever 200. Lumen 207 can accommodate a guide wire to assist with navigation through tortuous anatomy and stabilization in a vessel lumen during a clot retrieval procedure (as described below). Alternatively, lumen 207 can accommodate a distal protection device as known in the art, which can be used to catch or trap emboli in a procedure with clot retriever 200 when treating or inhibiting ischemic stroke caused by a partially or severely calcified clot. In the embodiment of FIGS. 3A-3E, the inner diameter (ID) of the clot retriever 200 (e.g., lumen 207) measured in a direction orthogonal to elongate central axis 231, can be approximately 0.0144 inches (0.3657 mm). In other embodiments, the ID of lumen 207 of the clot retriever 200 may range between 0.002 inches (0.0508 mm) to 0.020 inches (0.508 mm).

Optionally, and as shown in FIGS. 3D and 3E, clot retriever 200 can include a layer of material, e.g., a polymeric material, within (212) and/or external (214) to lumen 207 to increase the lubricity and reduce friction between the clot retriever and delivery assembly components (e.g., guide wire passing through lumen 207, or exterior surface of clot retriever passing through a delivery catheter lumen and/or blood vessel). The material layer(s), e.g., polymeric material layer(s), can comprise homopolymers, copolymers, or polymer blends containing, for example, polyamides, polyurethanes, silicones, polyolefins (e.g., polypropylenes, polyethylenes), fluoropolymers (e.g., FEP, TFE, PTFE, ETFE), polycarbonates, polyethers, PEEK, PVC, and other polymer resins. The layer thickness can range from approximately 0.0005 inches to 0.003 inches. In addition, the clot retriever embodiments can include hydrophilic coatings commonly known in the art to further increase the lubricity and navigability of the device through the delivery assembly 300 components within the patient.

While the cuts 210 generally can pass through the wall of the tubular body portion 203, as shown, for example, in FIG. 3E, the cuts 210 may or may not pass through the material layers 212 and/or 214, depending on the dimensions of the cuts and/or thickness of the material layers.

The patterns of the cuts 210 can be achieved by laser cutting the body portion 203 while rotating the body at a selected angle as the laser and body move with respect to one another. For example, with a laser oriented orthogonal to the longitudinal axis of the body 203 and with a fixture capable of holding body 203 while rotating and advancing the body 203 relative to the laser, the laser can be activated and deactivated to form specific cut patterns in clot retriever tubular body 203. FIGS. 3G, 3J, 3M, 3P, and 3S depict exemplary cut patterns in a two dimensional view of their respective tubular elongated bodies 203 of clot retrievers 200 shown in FIGS. 3F, 311, 3K, 3N, and 3Q, respectively. In the embodiments of FIGS. 3F-3M, the laser cutting of the body portion 203 creates 1.5 cuts 210 per rotation of the body, having a cut balance of about 210° of rotation of body 203 with laser on, and then 30° of rotation of body 203 with laser off. In the embodiments of FIGS. 3N-3P, the laser cutting of the body portion 203 creates 2.5 cuts 210 per rotation, having a cut balance of about 116° of rotation with laser on, followed by 28° of rotation with laser off. In the embodiments of FIGS. 3Q-3S, the laser cutting of the body portion 203 creates 2.5 cuts 210 per rotation, having a cut balance of about 116° on, 28° off.

Further, while the pitch of the cut pattern is approximately 0.0070 inches (0.1778 mm) in the embodiments of FIGS. 3F-3P, each cut 210 may have a variety of widths; for example, 0.0010 inches (0.0254 mm) (FIGS. 3F-3G), 0.0022 inches (0.05588 mm) (FIGS. 3H-3J), 0.0049 inches (0.12446 mm) (FIGS. 3K-3M) or 0.0039 (0.09906 mm) (FIGS. 3Q-3S). In the embodiment of FIGS. 3Q-3S, each cut 210 has a width of 0.00399 inches (0.10134 mm) and is oriented orthogonal to the tube's longitudinal axis, illustrating a zero-pitch pattern.

It should be appreciated that the above disclosed units are examples of useful dimensions, angles, and properties of the cuts 210 and their patterns, which are not intended to limit the scope of embodiments of clot retriever 200 disclosed herein.

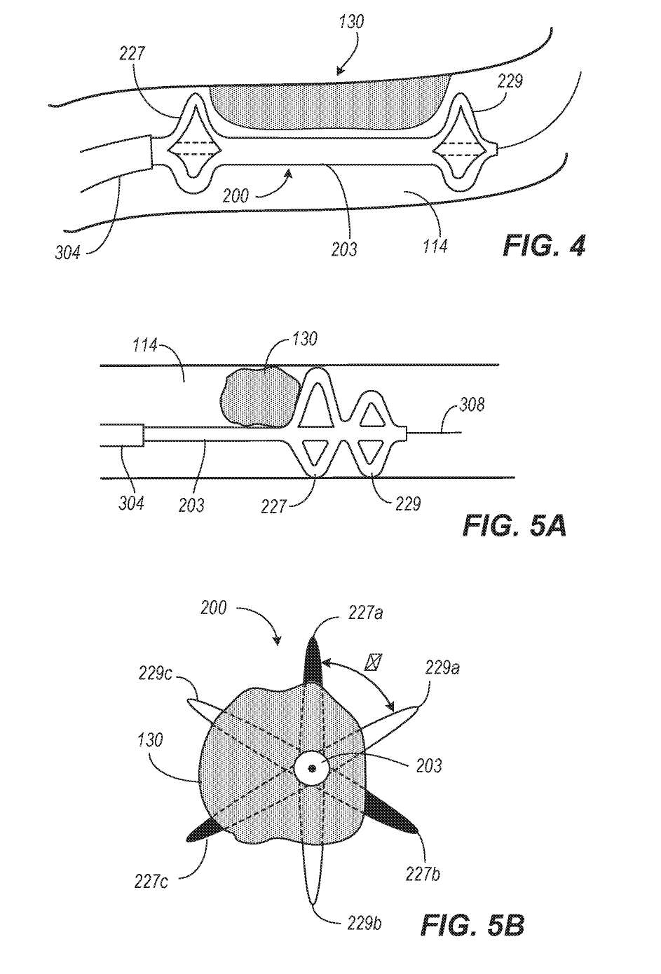

FIG. 4 depicts an example of a clot retriever 200 that has been deployed from a delivery catheter 304 to engage a calcified clot 130 disposed in a vessel 114. As shown in FIG. 4, this device has two clot engaging members 227 and 229, which are deployed partially proximal and distal of clot 130, respectively, to engage the clot between the engaging members and along the body portion 203 of the device. Clot 130 engaged by clot retriever 200 as depicted in FIG. 4 can be withdrawn into delivery catheter 304, partially or completely, to remove clot 130 from the patient's vasculature thereby restoring blood flow to the previously occluded artery.

FIG. 5A depicts another embodiment of a clot retriever 200 engaging a clot 130 in a vessel 114. Guide wire 308 has been advanced through a lumen of the clot retriever 200 (as described below) that has been deployed from delivery catheter 304. Clot retriever 200 includes two clot engaging members (deformable ribs) 227, 229 distal to a body portion 203 of the device. In this embodiment, each clot engaging member comprises a multiple rib, e.g., three-rib, configuration, and the ribs of the respective clot engaging members can be offset with respect to each other by an angle ϕ of approximately 60 degrees, in the three-rib embodiment as shown in the FIG. 5B perspective view of clot retriever 200. Clot retriever 200 can be deployed distal to a calcified clot 130 in a vessel, and withdrawn proximally in its deployed configuration to engage the clot for removal from the patient. The ribs of the respective clot engaging members trap clot 130 along device 200 as the clot retriever is pulled proximally through the vessel, and clot 130 engaged by clot retriever 200 can be pulled proximally, toward or into delivery catheter 304, for withdrawal from the patient. Other embodiments of clot retriever 200 can include different configurations of a multiple rib clot engaging member (e.g., 2, 4, 5, or more ribs on a clot engaging member).

Embodiments of clot retriever 200 with one or more clot engaging members configured to deploy distally of a clot 130 (e.g., as shown in FIG. 5A) are advantageously suited to treat ischemic stroke resulting from partially or severely calcified clots. In some circumstances, if such clots are detected before a stroke occurs, they can be removed to inhibit or prevent the occurrence of a stroke. Such clots are hardened relative to fresh thrombus. Existing mechanical thrombectomy devices typically collapse or simply fail to expand into calcified clots. With one or more clot engaging members (each with three or more ribs) deployed in a vessel distal of a calcified clot (e.g., as shown in FIG. 5A), clot retriever 200 can be withdrawn proximally through the vessel to engage and drag clot 130 to and optionally into the distal end of delivery catheter 130 for removal from the patient.

Embodiments of clot retriever 200 can be used with aspiration systems to remove calcified clots and further minimize the risk that dislodged clot particles travel distally through the patient's vasculature during the procedure. For example, the clot retriever can be deployed from an ACE™ 68 Reperfusion Catheter or Apollo System Catheter, both available from Penumbra, Inc. (Alameda, Calif.). Alternatively, clot retriever 200 can be deployed from a delivery catheter as described herein, while also using an aspiration system during the clot retrieval procedure. The suction provided by aspiration systems when used in conjunction with clot retriever 200 helps prevent unwarranted distal migration of the clot or clot particles through the patient's vasculature.

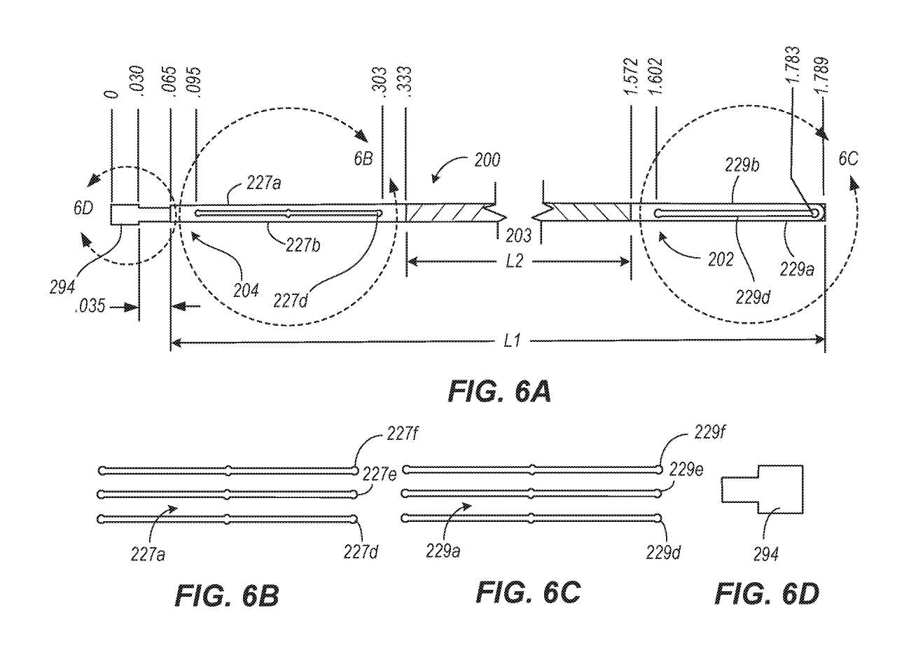

As shown in FIGS. 6A-6C, the clot engaging members 227 and 229 are formed by two, three, or more concentric parallel or radially spaced cuts 227d, 227e, and 227f, and 229d, 229e, and 229f along the length of the respective proximal 204 and distal 202 portions of the clot retriever body 203, forming the deformable elements, e.g., ribs, 227a, 227b, and 227c and 229a, 229b, and 229c, respectively. The deployed configuration of the clot engaging members provides sufficient resistive force to engage a calcified clot in the vasculature, while maintaining a curved, atraumatic outer profile to minimize the risk of damaging the arterial wall during deployment and clot retrieval.

In addition, FIG. 6A illustrates exemplary dimensions of a nitinol hypo-tube forming an embodiment of clot retriever 200. Dimensions referenced in FIG. 6A, are provided in inches, although it should be appreciated that such dimensions are examples only and not intended to limit the embodiments of clot retriever 200. The proximal portion 204 of clot retriever 200 depicted in FIG. 6A shows how a multiple rib clot engaging member 227 is formed by removing (e.g., cutting away) material from the hypo-tube between them indicated by the area of cuts 227d, e, fin FIG. 6B. Similarly, distal portion 202 includes a multiple rib clot engaging member 229 shown in FIG. 6A formed by removing material from the hypo-tube indicated by the area of 229d, e, f of FIG. 6C. As shown in the specific example illustrated in FIG. 6A, the overall length of the depicted embodiment clot retriever 200 in its constrained delivery configuration is 1.724 inches (i.e., distance between proximal end of proximal portion 204 and distal end of distal portion 202 represented by L₁ in FIG. 6A). Alternatively, the overall length of the device in a compressed or constrained delivery configuration can range from about 0.5 inches to 2.0 inches. The length of body portion 203 of the clot retriever embodiment shown in FIG. 6A is 1.239 inches; however, in other embodiments (e.g., in the embodiment of FIG. 11) of clot retriever 200, body portion 203 can range from about 0.25 inches to 1.5 inches or more. The outer diameter of the clot retriever 200, including the outer diameter of one or more clot engaging members, in a constrained delivery configuration can range from about 0.012 inches to 0.026 inches.

FIGS. 6A-C illustrate exemplary patterns and dimensions of cuts 227d, 227e, and 227f in the respective proximal portion 204 (FIG. 6A-B) and cuts 229d, 229e, and 229f in the distal portion 202 (FIGS. 6A and 6C) to form the proximal and distal clot engaging members 227 and 229 of clot retriever 200. It should be appreciated that the patterns and dimensions of the cuts 227d, 227e, and 227f in the proximal portion 204 may be similar or dissimilar from the patterns and dimensions of the cuts 229d, 229e, and 229f in the distal portion 202. Each deformable element 227a and 229a has respective hinge-like points 237a and 239a (e.g., living hinge, joint, or the like). As shown in FIG. 2, the hinge-like points 237a and 239a are configured to move radially outward from the axis of the clot retriever 200 in a hinge-like fashion, allowing the ribs 227a and 229a to be disposed radially outwardly so that the clot retriever 200 engages (e.g., traps, ensnares, or drags) the clot in or adjacent to body portion 203 between the expanded clot engaging members 227, 229. As shown in FIG. 2, the hinge-like points 237a and 239a have a curved, atraumatic profile to minimize damage to the arterial walls when deployed in a patient's vessel. The depicted patterns and dimensions of ribs 227a, 227b, and 227c and 229a, 229b, and 229c can also be used to configure embodiments of the clot retriever with one or more clot engaging member(s) disposed distal of body section 103, for example, two distal clot engaging members as shown in FIGS. 5A and 5B.

While the clot retriever 200 embodiments of FIGS. 2, 4, and 6A-6D include two clot engaging members each comprising three ribs, it should be appreciated that the depicted embodiments represent only examples of possible configurations and are not intended to limit the scope of embodiments of clot retriever 200 disclosed herein. For example, alternate embodiments of clot retriever 200 can include a different number of clot engaging members (e.g., 1, 3, 4, or more) on the device. Further, one or more of the clot engaging members may comprise a different number of ribs than depicted in FIGS. 2, 4, 6A-6D (e.g., 2, 4, 5, or more ribs on a clot engaging member) and different members can have the same or different number of ribs than other members. In addition, two or more clot engaging members on a clot retriever 200 can have different expanded diameters. For example, with reference to FIG. 5A, a distal clot engaging member can have a larger deployed diameter than the diameter of the more proximal clot engaging member (e.g., 15% larger diameter, 25% larger diameter, 35% larger diameter or more).

In some embodiments, clot retriever 200 can include an interlocking element 294 (e.g., clasp) arranged around a transition portion 201 and coupled to the proximal portion 204 of the clot retriever 200 (see, e.g., FIGS. 6A and 6D). The interlocking element 294 is configured to engage and disengage with an interlocking element coupled to the distal portion of the delivery assembly (e.g., delivery guide wire or pusher element 230, not shown) for deployment of the clot retriever 200 at the target site. FIG. 6D illustrates an example of a pattern used for laser cutting a tubular portion of super-elastic material to form an embodiment of the interlocking element 294.

The clot retriever 200 embodiments of FIGS. 2, 4, and 9 include clot engaging members 227, 229 oriented similarly along the longitudinal axis of the device (e.g., along the axis of the body portion 203 represented by L₂ in FIG. 2). The orientation of the clot engaging members can differ in other embodiments of clot retriever 200. As shown in FIGS. 7A, 7B the center portion of clot engaging member 229 is angled or bent approximately 90 degrees with respect to the center portion of clot engaging member 227. Of course, other orientations between the clot engaging members 227, 229 of the device are possible. Such configurations can facilitate engagement and retrieval of calcified clots in tortuous vasculature, e.g., particularly where the calcified clot is adhered in or along a curved portion of an arterial wall, such as one or more of the vessels described in connection with FIG. 1.

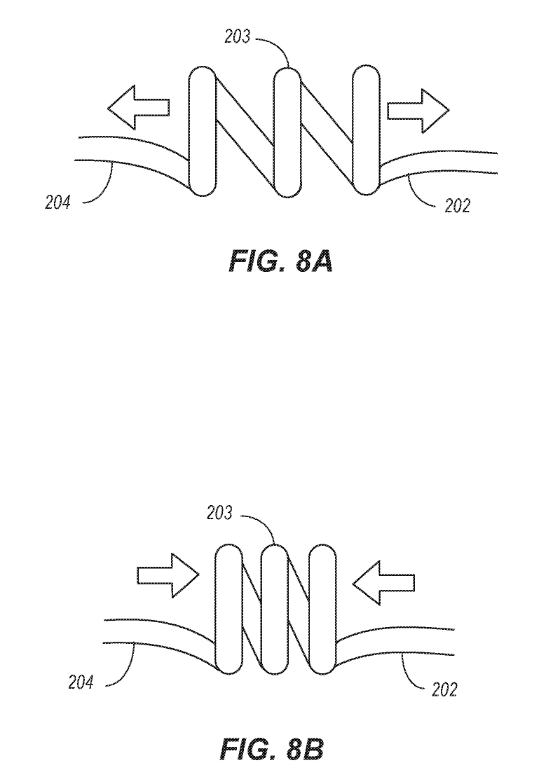

Clot retriever 200 can have a body portion 203 with a biased or preformed coil shape that tensions clot engaging members 227, 229 toward each other, to secure clot 130 in device 200. FIG. 8A shows one embodiment of a coiled body 203 of a clot retriever 200. As depicted by the arrows in the figure, the coil can be straightened and compressed for delivery via catheter to a target location in a patient's arterial vasculature. After deployment about a clot 130 in a vessel, the coil in body 203 resumes its helical configuration and pinches clot engaging members toward the center of device 200 (e.g., direction of arrows in FIG. 8B), further securing the engaged clot 130 within clot retriever 200 for withdrawal from the patient. While the coil shape of body 203 shown in FIGS. 8A-8B includes three coils, other configurations are possible as long as they provide sufficient tensioning force to pinch or secure a clot between the respective clot engaging members 227, 229 (e.g., single coil in body 203 as depicted in FIG. 7B). Further, in embodiments of clot retriever 200 with only distal clot engaging members, the biased or preformed coil shape of body 203 can provide additional elasticity to the device, which can be useful when engaging and retrieving calcified clots from blood vessel walls.

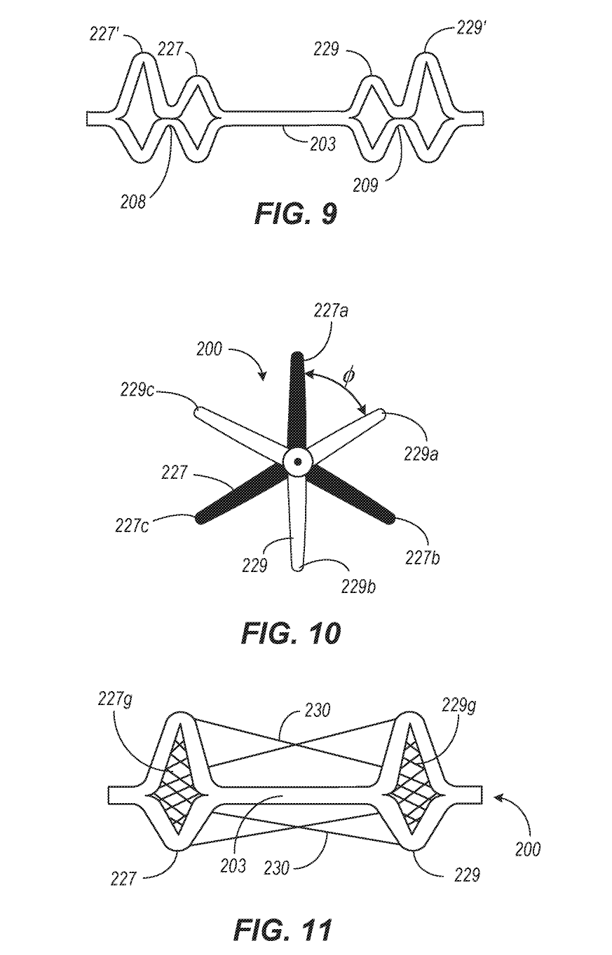

While the clot retriever 200 described in connection with FIGS. 2, 4, 6A-6D, includes two clot engaging members, other device configurations with a different number of clot engaging members are possible (e.g., 1, 3, 4, or more clot engaging members). FIG. 9 depicts one embodiment of a clot retriever 200 that includes four clot engaging members: 229 and 229′ on the distal portion 202 of the device, and 227 and 227′ on the proximal portion 204 of the device. As shown in FIG. 9, the diameter of the inner clot engaging members 227, 229 in an expanded configuration is approximately 50% of the diameter of the outer clot engaging members 227′, 229′. In alternate embodiments, the diameter of inner clot engaging members 227, 229 in an expanded configuration can range from about 20% to 200% of the expanded diameter of the outer clot engaging members 227′, 229′ in an expanded configuration. Clot retriever 200 of FIG. 9 includes a proximal intermediate region 208 between a proximal-most clot engaging member 227′ and clot engaging member 227. The distal portion of the device includes a distal intermediate region 209 between a distal-most clot engaging member 229′ and clot engaging member 229. The length of each of proximal intermediate region 208 and distal intermediate region 209, as well as the length of body 203, can be optimized for more effective engagement and retrieval of calcified clots from a patient's vasculature.

The ribs 227a, 229a of each clot engaging member can be oriented similarly with respect to each other (e.g., the same orientation along the longitudinal axis of clot retriever 200), or can have different orientations with respect to each other. FIG. 10 shows an end view of a clot retriever 200 with clot engaging members 227, 229. Each clot engaging member contains three ribs, e.g., ribs 227a, 227b, and 227c of clot engaging member 227 and ribs 229a, 229b, and 229c of clot engaging member 229. The orientation of the respective ribs of each clot engaging member 227, 229 differs by approximately 60 degrees, as represented by the angle ϕ shown in FIG. 10. It should be appreciated, however, that the depicted angle and orientation between the ribs of the respective clot engaging members 227, 229 are examples only, and are not intended to limit the embodiments of clot retriever 200 disclosed herein.

Clot retriever 200 can have additional features to facilitate engaging and retrieving calcified clots from a patient's vasculature. FIG. 11 depicts an embodiment of clot retriever 200 with a proximal clot engaging member 227, and a distal clot engaging member 229. Each clot engaging member 227, 229 includes a fine mesh structure 227g, 229g between the respective ribs 227a, 229a of each clot engaging member. Clot retriever 200 also includes wires or filaments 230 extending between the respective ribs 227a, 229a of each clot engaging member 227, 229. The mesh 227g, 229g and wires 230 provide additional contact points between the clot retrieval device 200 and clot 130 to further secure and engage the clot for removal from the patient's vasculature. In addition, the mesh and wire features can trap pieces of clot that may break from clot 130 during the clot retrieval procedure to minimize or prevent the risk of emboli in new regions of the patient's vasculature. Mesh and/or wire features can also be used in embodiments of clot retriever 200 that include only distal clot engaging member(s).

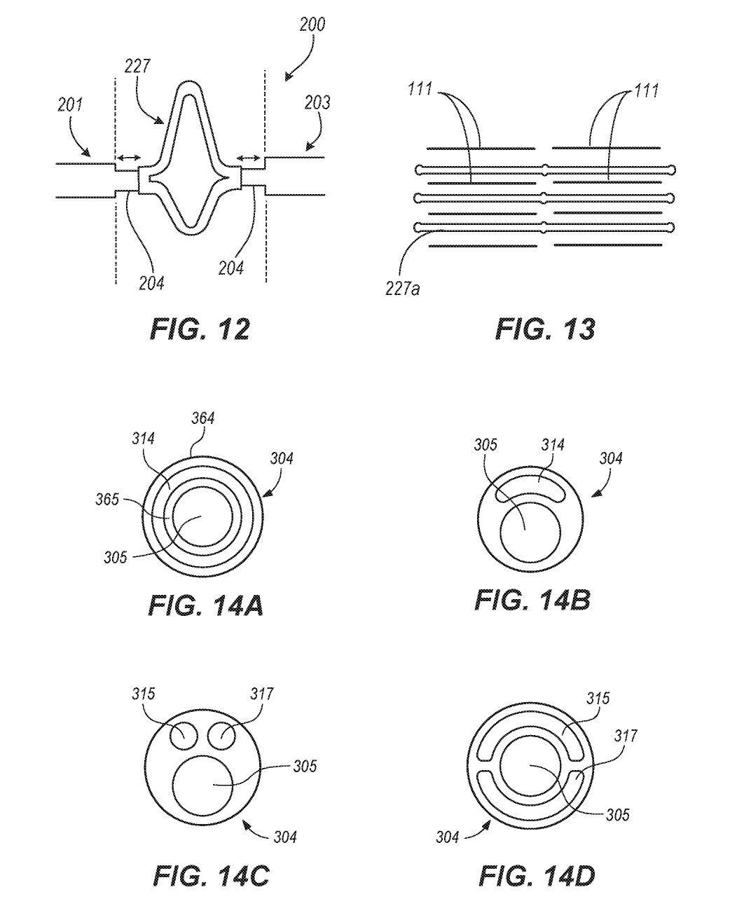

Further, clot engaging members 227, 229 can be configured to move along the longitudinal axis of clot retriever 200, further facilitating engagement and retrieval of calcified clots from a patient's vasculature. FIG. 12 depicts a clot engaging member 227 comprising a three-rib clot engaging member located on proximal portion 204 of a clot retriever 200. The outer diameter of the clot retriever in proximal portion 204 is reduced (e.g., region between dotted lines in FIG. 12) compared to the outer diameter of the transition portion 201 and body portion 203 of the device 200. The clot engaging member 227 of FIG. 12 includes a central lumen configured to slidably accommodate proximal portion 204 of the clot retrieval device, and allows the engaging member 227 to travel proximally and distally between the transition portion 201 and body portion 203 (e.g., horizontally, as indicated by the bi-directional arrows on either side of clot engaging member in FIG. 12). The clot engaging member can be configured to travel along the longitudinal axis of clot retriever from about 1 mm to 1 cm or more. A moveable clot engaging member 229 can also be disposed on the distal portion 202 of the clot retrieval device 200.

A clot retriever 200 with a moveable proximal clot engaging member 227 can advantageously secure clot 130 on the device 200 for withdrawal from the patient's vasculature. For example, after deployment about clot 130 in a patient's vessel (e.g., as depicted in FIG. 4), clot 130 engaged by clot retriever 200 is withdrawn proximally, towards and/or into the open distal end of delivery catheter 304. As this occurs, proximal engaging member 227 can slide distally along proximal portion 204 of the clot retrieval device as the proximal engaging member encounters the distal end of the delivery catheter and the clot 130 and distal engaging member move proximally, thereby pinching or further securing the clot between the engaging members of the device 200. The moveable clot engaging member feature can also be used in embodiments of clot retriever 200 that include only distal clot engaging member(s).

It should be appreciated that the features and configurations of the clot engaging member 227 and clot retriever 200 of FIG. 12 are examples only, and not intended to limit the scope of embodiments of a clot retriever with slidable or moveable clot engaging members.

For example, each clot engaging member 227, 229 can be configured from a dedicated concentric shaft, with a distal clot engaging member 229 configured to telescope or deploy through the shaft of the proximal clot engaging member 227. In this configuration, the respective clot engaging members can slide relative to each other to engage a clot for withdrawal from the patient's vasculature.

Radiopaque markings or coatings can be incorporated on portions of clot retriever 200 to assist with navigation and deployment in the arterial vasculature. The radiopaque markings may be placed on one or more of the following locations along the device 200: along one or more ribs (e.g., 227a) of the clot engaging member (e.g., markings 111 on the ribs or ribs as depicted in FIG. 13), along the middle portion 203 of the device, along the pusher element 230, and/or at the junction of the pusher element 230 and clot retriever 200 in the transition portion or zone 201, or on the proximal portion 204 and/or distal portion 202 of the clot retrieval device. For example, at any such portion or junction, a semi-circle piece or half-band of radiopaque material can be coupled to or incorporated within the clot retriever to help the operator visualize the location and/or orientation of the device in the vasculature.

Clot retriever 200 can be advantageously used to remove calcified clots from a patient's vasculature based, in part, on its overall flexibility, the radial expansion force of the clot engaging members as the device self-expands from its compressed, e.g., constrained, delivery configuration into its non-constrained, self-expanded deployed configuration, and sufficiently robust construction of the ribs of the clot engaging members of the device. Cuts 210 and the examples of cut patterns described with FIGS. 3A-3S create a highly flexible clot retriever 200 that can be delivered to and navigated through tortuous anatomy to access the calcified clot. Super-elastic materials (e.g., nickel-titanium alloys such as nitinol) add to the overall device flexibility, while preserving sufficient column strength to engage and remove calcified clots bonded to the walls of the patient's vasculature where clot retrieval devices typically fail.

In addition, the configuration of the clot engaging member(s) including the dimensions of the deployed, expanded ribs provide sufficient resistive force such that the clot engaging members do not break or resume a constrained delivery configuration when engaging a hardened, calcified clot 130 in a vessel. The exterior portions of the clot engaging members 227, 229 (e.g., hinge-like points 237a, 239a shown in FIG. 2) have a curved, atraumatic profile to minimize damage to the arterial walls when deployed in a patient's vessel. Further, in embodiments with a coiled or other body section 203 configured to tension clot engaging members toward each other, severely calcified clots can be secured in the device and removed from the patient's vasculature.

FIGS. 14A-D illustrate cross-sectional views of examples of delivery catheter embodiments 304 and 304″ for delivering the clot retriever 200 into a target site of a patient, and removing an engaged clot 130 and clot retriever 200 from the patient's vasculature. FIG. 14A depicts a cross-sectional view of a delivery catheter 304 comprising a tubular interface having an outer tubular member 364 and an inner tubular member 365 coaxially disposed within the outer tubular member 364. The coaxial tubular interface of the catheter 304 comprises the lumen 305 (or, alternatively, lumen 314) configured to deliver the clot retriever 200 into the target site, and the lumen 314 (or, in the alternative, lumen 305) configured for advancement of guide wires to the vasculature and/or any other suitable function (e.g., aspiration to draw clot 130 or pieces thereof into the delivery catheter for withdrawal from the patient).

FIG. 14B depicts a cross-sectional view of another embodiment of delivery catheter 304 comprising lumen 305 to deliver clot retriever 200. Lumen 314 can accommodate a guide wire to facilitate access and navigation of the delivery catheter through tortuous arterial vasculature. FIGS. 14C-D depict cross-sectional views of further embodiments of delivery catheter 304 comprising a lumen 305 configured to deliver the clot retriever 200 into the target site, and two additional lumens, lumen 315 and lumen 317, for guide wires or other additional tools. It should be appreciated that any other configuration of the delivery catheter and lumens suitable for delivering the clot retriever 200 into the target site may be used. For example, standard micro catheters (e.g., 0.027″, 0.021″, and 0.021″) used in conjunction with one or more guide catheters (e.g., 7Fr or 6Fr), intermediate guide catheters (e.g., Navien Intracranial Support Catheter from Medtronic, Inc. of Minneapolis, Minn.) and/or distal access catheters (e.g., Sofia Distal Access Catheter from MicroVention, Inc. of Tustin, Calif.) can be used in procedures to remove calcified clots where embodiments of clot retriever 200 are deployed from the micro catheter, intermediate guide catheter, or distal access catheter.

Lumens of the catheter embodiments depicted in FIGS. 14A-14D can be configured to conform to the various delivery assembly 300 elements used with such catheters. Lumen 314 of delivery catheter 304″ depicted in FIG. 14B comprises a crescent shaped profile that opens into distal end 350. In other embodiments, the profile of all or a portion of lumen 314 can be configured to more closely match the exterior profile of a guide wire 318. Conformed catheter lumens can eliminate the risk that the element passing through inadvertently changes orientation or trajectory within the catheter during the clot retriever procedure. In addition, any combination of conformed lumens can be used with or in place of the circular and crescent lumen 314 embodiments shown in FIG. 14A-D. It will be appreciated by those of skill in the art, however, that certain lumen 314 configurations (e.g., crescent lumen versus rectangular lumen of equal size) can conserve more cross-sectional area of the catheter to accommodate other lumens and componentry.

The lumens of the delivery catheter 304 embodiments depicted in FIGS. 14A-14D and disclosed elsewhere in this application can include a liner to increase the lubricity of the delivery assembly 300 and reduce friction between the specific catheter lumen and delivery system components delivered through such lumen. The catheter liner may comprise homopolymers, copolymers or polymer blends containing polyamides, polyurethanes, silicones, polyolefins (e.g., polypropylenes, polyethylenes), fluoropolymers (e.g., FEP, TFE, PTFE, ETFE), polycarbonates, polyethers, PEEK, PVC, and other polymer resins. The liner thickness can range from approximately 0.0005 inches to 0.003 inches. In addition, the catheter embodiments can include hydrophilic coatings commonly known in the art to further increase the lubricity and navigability of the delivery assembly 300 components within the patient.

A variety of different imaging methods can be used to ensure accurate positioning, navigation, deployment, and withdrawal of the clot retriever 200, guide catheter 320, guide wire 302, micro catheter 304, and/or micro guide wire 308. Examples of suitable imaging methods include biplane fluoroscopy, digital subtraction angiography with road mapping technology, arterial or venous angiography with road mapping technology, 3D-rotational angiography or venography (3DRA or 3DRV), and cone-beam computed tomographic angiography or venography (CBCTA or CBCTV). Both 3DRA/V and CBCTA/V enable volumetric reconstruction showing the relationship among the bony anatomy, the arterial anatomy, clot retriever 200, and the radiopaque catheters and guide wires used for clot retriever deployment and thrombectomy. The methods of navigating, positioning, deploying, and withdrawing the clot retriever 200 comprise imaging the clot retriever 200 in the patient during the procedure.

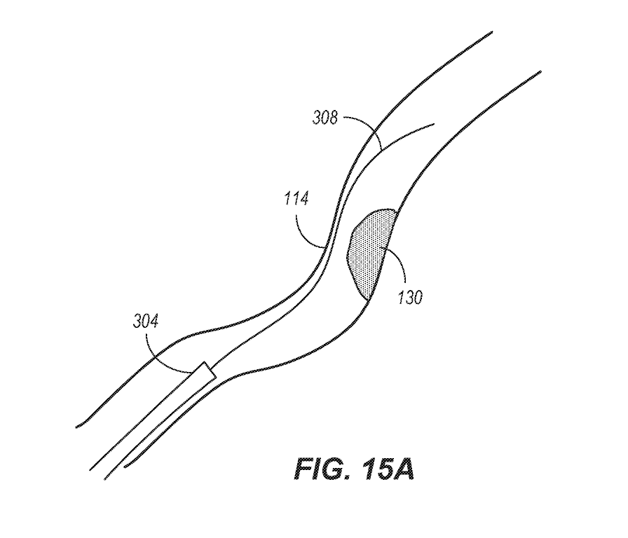

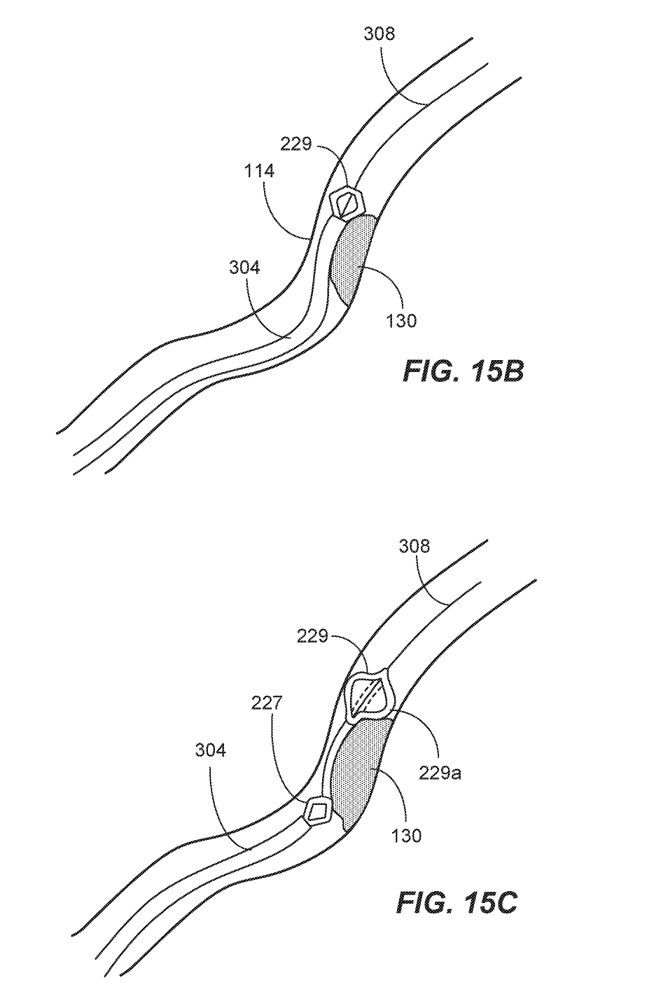

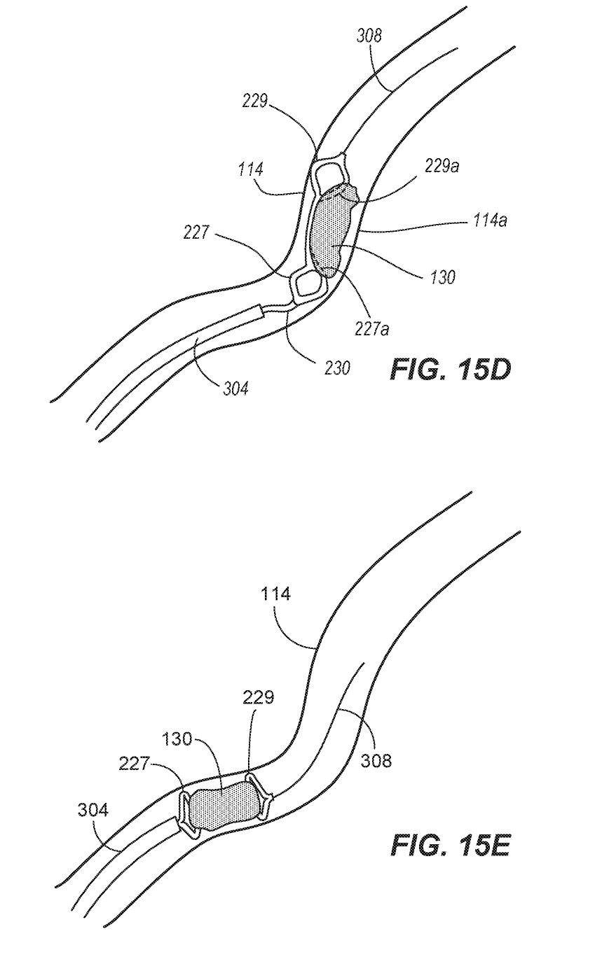

FIGS. 15A-15E depict an example of a procedure for deploying a clot retriever 200 to engage and withdraw a calcified clot 130 from a patient's middle cerebral artery 114, thereby restoring arterial blood flow after an acute ischemic stroke. The following description references a delivery catheter 304 and delivery guide wire 308 that a clinician has navigated from an access point in the patient's vasculature at the femoral artery, through arterial vasculature to the internal carotid artery, and further into MCA 114. Delivery catheter 304 includes a lumen (e.g., as described in connection with FIG. 14A-14D), through which clot retriever 200 can be delivered to a target location within the patient's vasculature to engage a clot; clot retriever 200 and clot 130 can be subsequently withdrawn into, partially or completely, the delivery catheter for removal from the patient. Other components of a delivery system 300, such as one more guide wires 302 and/or guide catheters 320, intermediate guide catheters, or distal access catheters (e.g., guide, intermediate guide, and distal access catheters all larger than delivery guide wire 308 and/or delivery catheter 304), can also be used in the procedure to facilitate navigation and delivery of a clot retriever 200 and/or delivery catheter 304 to a clot in the arterial vasculature.

FIG. 15A depicts a guide wire 308 that has been advanced through the patient's ICA 112 (not shown here, but see FIG. 1), and advanced further distally beyond a severely calcified clot 130 that has migrated to a location in the lumen of MCA 114. A delivery catheter 304 has been advanced to a location in MCA 114 proximal of clot 130 via guide wire 308. A clot retriever 200 and integrated pusher element 230 have been loaded in the delivery catheter such that the distal portion of the clot retriever is disposed slightly proximal of the distal opening in the delivery catheter. The clinician will continue to advance the delivery catheter 304, with clot retriever 200 disposed inside the catheter, distal of the clot 130 in MCA 114.

In the next step of the procedure, as shown in FIG. 15B, the delivery catheter 304 has been advanced distal of clot 130, and the clinician has started to deploy clot retriever 200 to engage clot 130. The clinician can use pusher element 230 to advance the clot retriever 200 distally from the open distal end of delivery catheter 304, and/or can use pusher element 230 to hold clot retriever 200 stationary while withdrawing delivery catheter 304 proximally. Distal clot engaging member 229 of the device 200 has been partially deployed in the MCA from the distal opening of delivery catheter 304, but remains slightly constrained by the lumen of delivery catheter 304 as depicted in FIG. 15B.

In the next step, the clinician withdraws delivery catheter 304 proximally (e.g., while holding pusher element stationary to maintain the location of clot retriever 200 in the patient's vasculature), and/or advances pusher element 230 distally while holding delivery catheter 304 stationary. As shown in FIG. 15C, the distal clot engaging member 229 is fully deployed such that at least one rib 229a is in direct contact with clot 130. The body portion of clot retriever 200 has emerged from the delivery catheter, and the proximal clot engaging member 227 has partially deployed in the MCA 114.

Continuing to the next step of the procedure, FIG. 15D depicts a fully deployed clot retriever 200 engaging clot 130. The ribs 227a, 229a of the proximal and distal clot engaging members have embedded within clot 130 in the vessel; alternatively, and depending on the severity of calcification in clot 130, the ribs of the respective clot engaging members may only contact the clot without embedding in the clot, thereby pinching or trapping the clot along body portion 203 of the device between the respective clot engaging members. With clot 130 trapped between the clot engaging members of the device, the clinician has started to pull the clot away from its resting point in the patient's arterial vasculature, the inner wall 114a of MCA 114 as shown in FIG. 15D. Thereafter, the clinician continues the procedure by withdrawing engaged clot 130 and clot retriever 200 from the patient's vasculature.

As shown in FIG. 15E, the engaged clot 130 and clot retriever 200 have been withdrawn (proximally) to the distal opening of delivery catheter 304. Depending on the size and degree of calcification of the clot, the clot retriever 200 and clot 130 may be withdrawn into the delivery catheter lumen. Alternatively, in the case of larger and/or harder clots, the engaged clot 130 and clot retriever 200 remain withdrawn to the distal end of delivery catheter 304, while the delivery assembly 300 (e.g., delivery catheter 304, clot retriever 200, and clot 130) is withdrawn from the patient, thereby restoring arterial blood flow in the MCA 114.

It should be appreciated that the method disclosed in FIGS. 15A-E may include any steps and features disclosed herein, including steps and features disclosed in connection with different embodiments of clot retriever 200, in any combination as appropriate. For example, the engaged clot 130, clot retriever 200, and delivery catheter 304 can be withdrawn, partially or completely, into a larger guide, intermediate guide, and/or distal access catheter before removal from the patient.

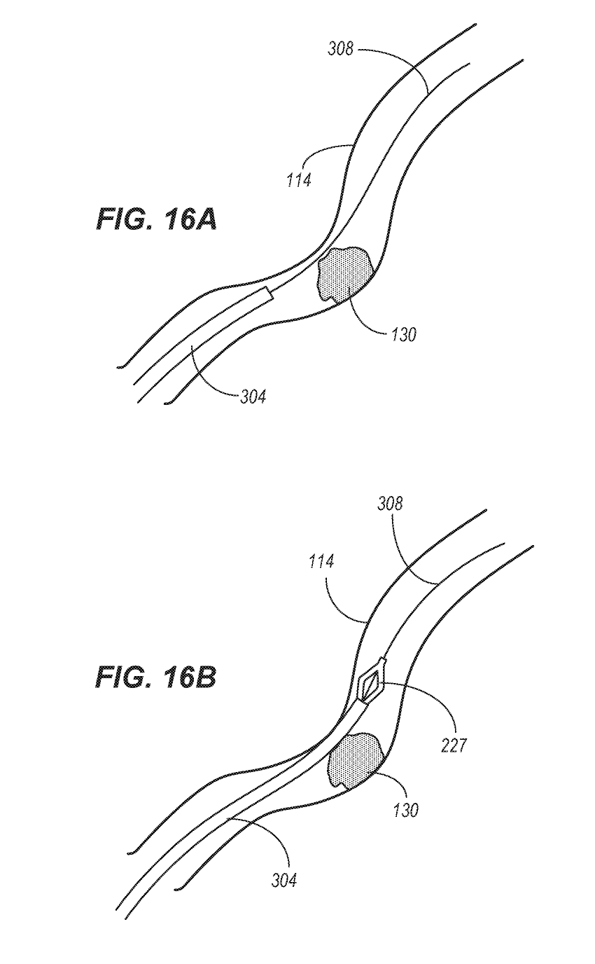

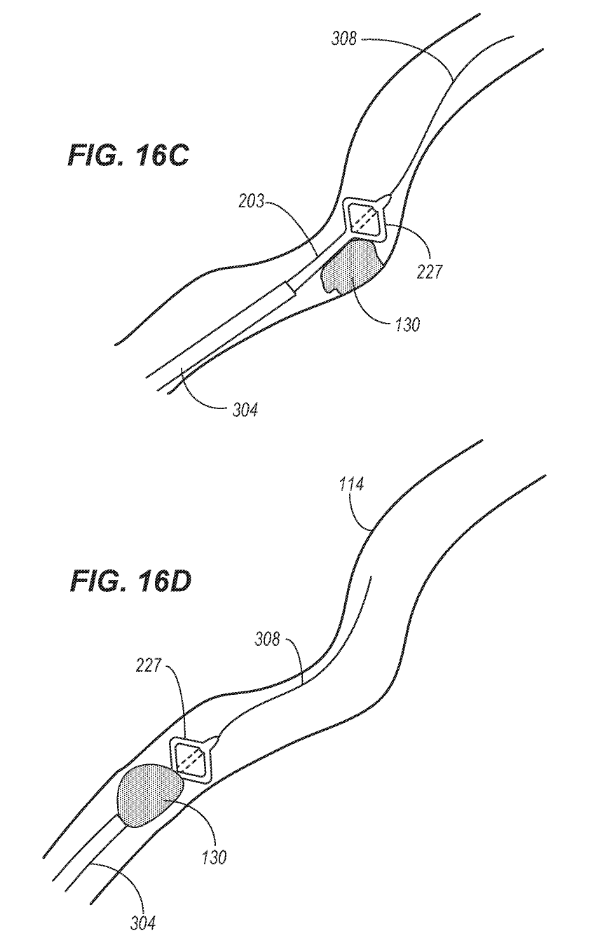

FIGS. 16A-16D depict another example of a procedure for deploying a clot retriever 200 to engage and withdraw a calcified clot 130 that has migrated to location in the lumen of the patient's middle cerebral artery 114. With respect to the following description, a clinician has advanced a delivery catheter 304 and delivery guide wire 308 from an access point in the patient's vasculature at the femoral artery, through arterial vasculature to the internal carotid artery, and further into MCA 114. Delivery catheter 304 includes a lumen, through which clot retriever 200 can be delivered to a target location within the patient's vasculature to engage a clot and subsequently withdraw such clot, partially or completely, into the delivery catheter. Other components of a delivery system 300, such as one or more guide wires 302 and/or guide catheters 320, intermediate guide catheters, or distal access catheters (e.g., guide, intermediate guide, and distal access catheters all larger than delivery guide wire 308 and/or delivery catheter 304), can also be used in the procedure to facilitate navigation and delivery of a clot retriever 200 and/or delivery catheter 304 to a clot in the arterial vasculature.

FIG. 16A depicts a guide wire 308 that has been advanced through the patient's ICA 112 (not shown here, but see FIG. 1), and advanced further distally beyond a severely calcified clot 130 present in the lumen of MCA 114. A delivery catheter 304 has been advanced to a location in MCA 114 proximal of clot 130 via guide wire 308. A clot retriever 200 and integrated pusher element 230 have been loaded and advanced distally in the delivery catheter lumen such that the distal portion of the clot retriever 200 is disposed slightly proximal of the distal opening in the delivery catheter. The clinician will continue to advance the delivery catheter 304, with clot retriever 200 inside the catheter, distal of the clot 130 in MCA 114.

In the next step of the procedure, as shown in FIG. 16B, the delivery catheter 304 has been advanced distal of clot 130, and the clinician has started to deploy clot retriever 200 in the vessel 114 distal to clot 130. The clinician can use pusher element 230 to advance the clot retriever 200 distally from the open distal end of delivery catheter 304, and/or can use pusher element 230 to hold clot retriever 200 stationary while withdrawing delivery catheter 304 proximally. Clot engaging member 227 of the device 200 has been partially deployed in the MCA from the distal opening of delivery catheter 304, but remains slightly constrained by the lumen of delivery catheter 304 as depicted in FIG. 16B.

In the next step, the clinician withdraws delivery catheter 304 proximally (e.g., while holding pusher element 230 stationary to maintain the location of clot retriever 200 in the patient's vasculature), and/or advances pusher element 230 distally while holding delivery catheter 304 stationary to deploy clot retriever 200. As shown in FIG. 16C, the clot engaging member 227 is fully deployed in the vessel immediately distal of clot 130. The body portion 203 of clot retriever 200 has emerged from the delivery catheter and is contacting clot 130 in MCA 114.

Continuing to the next step of the procedure, FIG. 16D depicts the withdrawal of a clot retriever 200 and engaged clot 130 from the lumen of vessel 114. By withdrawing the deployed clot engaging member 227 proximally in the vessel in the direction of clot 130 (e.g., by pulling proximally on pusher element 230), the ribs of the clot engaging member have trapped clot 130 on the device and pulled the clot away from its resting point along the vessel wall. Withdrawing the clot retrieval device 200 further proximally in the vessel while maintaining the delivery catheter 304 stationary traps clot 130 between distal end of the delivery catheter 304 and the ribs of clot engaging member 227 as shown in FIG. 16D. Thereafter, the clinician continues the procedure by withdrawing the engaged clot 130 and clot retriever 200 from the patient's vasculature, thereby restoring blood flow to MCA 114.

It should be appreciated that the method disclosed in FIGS. 16A-D may include any steps and features disclosed herein, including steps and features disclosed in connection with different embodiments of clot retriever 200, in any combination as appropriate. For example, the engaged clot 130, clot retriever 200, and delivery catheter 304 can be withdrawn, partially or completely, into a larger guide, intermediate guide, and/or distal access catheter before removal from the patient.

### Other Embodiments

It is to be understood that while the technology has been described in conjunction with the detailed description, the foregoing description and Examples are intended to illustrate and not limit the scope defined by the appended claims. Other aspects, advantages, and modifications are within the scope of the following claims.

1. A system for removing a calcified clot from a blood vessel, the system comprising
a clot retrieval device comprising an elongated tubular body comprising a wall surrounding a central lumen and having proximal and distal ends, wherein the elongated tubular body comprises at least one set of two or more elongated longitudinal cuts through the wall to form a clot engaging member comprising two or more deformable ribs between the cuts, wherein a first set of elongated longitudinal cuts is arranged adjacent to the distal end of the elongated tubular body, and wherein the ribs comprise an elastic material configured to cause the ribs to self-expand radially outwardly from the body when the clot engaging member is in a non-constrained, deployed configuration and wherein the deployed, expanded ribs provide sufficient resistive force such that the ribs do not break or resume a constrained delivery configuration when engaging a hardened, calcified clot; and
a pusher element coupled to a proximal portion of the clot retrieval device and configured to enable a user to move the device proximally and distally within the blood vessel.

2. The system of claim 1, wherein the set of two or more elongated cuts comprises at least three elongated cuts to form a clot engaging member comprising three or more ribs between the cuts.

3. The system of claim 1, wherein the elongated tubular body comprises cuts arranged proximally of the clot engaging member to provide greater flexibility to the tubular body.

4. The system of claim 1, wherein the elongated tubular body comprises a second set of elongated cuts arranged proximally from the first set of elongated cuts to form a second clot engaging member, wherein a body portion separates the first and second sets of cuts.

5. The system of claim 1, wherein the elastic material comprises a metal or a metal alloy.

6. The system of claim 5, wherein the metal alloy comprises a shape-memory alloy and/or super-elastic alloy that returns to a predetermined shape when in a non-constrained, deployed configuration.

7. The system of claim 6, wherein the shape-memory alloy and/or super-elastic alloy comprises nickel and titanium.

8. The system of claim 1, wherein the elongated longitudinal cuts are arranged in parallel, are radially spaced apart at equal distances between them, or are both arranged in parallel and radially spaced apart at equal distances between them.

9. The system of claim 1, wherein the pusher element comprises a detachable wire connected to the elongated tubular body.

10. The system of claim 1, wherein the pusher element comprises a non-detachable wire connected to the elongated tubular body.

11. The system of claim 1, wherein the pusher element and the elongated tubular body are formed from a single piece of material.

12. The system of claim 4, wherein the elongated tubular body comprises a biased or preformed coil shape that tensions the first and second clot engaging members towards each other.

13. The system of claim 4, wherein the elongated tubular body comprises cuts arranged to provide greater flexibility to the body portion.

14. The system of claim 1, wherein exterior portions of the first and/or second clot engaging member(s) have a curved, atraumatic profile.

15. A method of removing a calcified clot from a blood vessel in a subject, the method comprising
guiding a delivery catheter through a blood vessel in the subject to a location in the blood vessel distally beyond the calcified clot, wherein the delivery catheter contains a clot removal system of claim 1 and wherein the at least one clot engaging member is in a constrained delivery configuration within the delivery catheter;
using the pusher element to advance the clot retrieval device partially out of the delivery catheter while maintaining the at least one clot engaging member in a constrained delivery configuration;
withdrawing the delivery catheter while maintaining the clot retrieval device in position within the blood vessel with the pusher element to advance the clot retrieval device out of the delivery catheter such that at least one clot engaging member self-expands into a non-constrained, deployed configuration;
manipulating the clot retrieval device to cause ribs of the at least one clot engaging member in the non-constrained, deployed configuration to contact the clot from a location distally beyond the clot; and
withdrawing the clot retrieval device from the location to pull the clot along with the at least one clot engaging member in the non-constrained, deployed configuration until the clot has been removed from the blood vessel.

16. The method of claim 15, further comprising
guiding a guide wire through the blood vessel in the subject to a location in the blood vessel distally beyond the calcified clot; and
guiding the delivery catheter along the guide wire to a location in the blood vessel distally beyond the calcified clot.

17. The method of claim 15, wherein withdrawing the clot retrieval device comprises pulling the clot retrieval device into the delivery catheter such that the clot is also pulled into the delivery catheter.

18. The method of claim 15, wherein withdrawing the clot retrieval device comprises pulling the clot retrieval device to a distal end of the delivery catheter such that the clot is secured between the distal end of the delivery catheter and one or more ribs of the at least one clot engaging member in the non-constrained, deployed configuration.

19. The method of claim 15, wherein the clot retrieval device comprises at least two clot engaging members and wherein the clot retrieval device is manipulated such that the first clot engaging member is deployed distally beyond the clot and the second clot engaging member is deployed proximally of the clot such that the clot is located between the first and second clot engaging members.

20. The method of claim 19, wherein the elongated tubular body of the clot retrieval device comprises a biased or preformed coil shape that tensions the first and second clot engaging members towards each other and the method further comprises manipulating the biased or preformed coil shape to cause the first and second clot engaging members to secure the clot between them.
